(19) United States
(12) Patent Application Publication
(10) Pub. No.: US 2026/0168954 A1
(43) Pub. Date: Jun. 18, 2026

(54) ELECTRODE FOR ELECTROCHEMICAL SENSOR

(71) Applicant: ELUCEDA LIMITED, Burnley Lancashire (GB)

(72) Inventors: Mark SAW, York Yorshire (GB); Ian Michael EASTWOOD, Rosendale Lancashire (GB)

(73) Assignee: ELUCEDA LIMITED, Burnley Lancashire (GB)

(21) Appl. No.: 19/125,241

(22) PCT Filed: Oct. 31, 2023
(86) PCT No.: PCT/GB2023/052836

§ 371 Date: Apr. 28, 2025

(30) Foreign Application Priority Data
Nov. 1, 2022 (GB) 2216218.4

(51) Int. Cl. G01N 27/327; G01N 33/543

(52) U.S. Cl. CPC G01N 27/3276; G01N 33/5438; G01N 2333/36

(57) ABSTRACT

The present invention relates to an electrode comprising a conductive element; and a coating layer formed on at least one surface of the conductive element, wherein the coating layer comprises a polymer having: (i) a conjugated system comprising at least two nitrogen atoms, and (ii) a hydrophobic region. The present invention also relates to a method of manufacturing such electrodes. The present invention further relates to a biosensor comprising such electrodes and to their use in the detection of chemical or biological analyte molecules.

### FIELD OF THE INVENTION

The present invention relates to an electrode, in particular, an electrode for a biosensor, and to a method of manufacturing such electrodes. The present invention further relates to a biosensor comprising such electrodes and to their use in the detection of chemical or biological analyte molecules.

### BACKGROUND OF THE INVENTION

A significant hurdle in the development of any biologically integrated device, such as a biosensor, is the ability to immobilise biomolecules onto a defined target surface. In particular, maximising the amount and activity of biological molecules immobilised in this way is essential for ensuring the sensitivity of such devices. In a biosensor, a biological element is immobilised on the surface of a physicochemical transducer for the detection of an analyte. The biological component, such as an enzyme, an antibody, a nucleic acid, a microorganism, interacts with the analyte, causing the transducer to produce a signal that may be detected. As a result of the high selectivity and affinity of the biological component, the development of biosensors is of considerable importance in areas such as clinical diagnostics, biomedical sector, bioreactor control and analytics, food production and analysis, bacterial and viral diagnostics, pharmaceutical analysis, industrial waste water control, environmental protection and pollution control, and toxic gas analysis.

In an electrochemical biosensor, the interaction of the biological component with the analyte results in an electrical signal that can be detected using various means, for example, by using amperometry, voltammetry or potentiometry. An electrode is employed as a solid support for immobilisation of a biomolecule and electron movement. The immobilisation of biomolecules onto an electrode surface can be executed by two principal means: active adsorption and passive adsorption. Active adsorption refers to the covalent attachment of a biomolecule to the electrode surface using different reactive groups (e.g., amine, thiol, carboxyl) on the interacting molecules. Active adsorption typically provides a strong interaction but can require complex methodology and also suffers from high sensitivity to contaminants and experimental conditions. As such, active adsorption often results in a precise but low yield attachment. Passive adsorption, on the other hand, involves directly exposing the electrode surface to the biomolecule and relying on interactions such as electrostatic, hydrophobic or hydrophilic interactions to govern adsorption, resulting in a non-covalent attachment. Passive adsorption is a more direct and user-friendly method but is subject to high variance between biomolecules and is not as robust as active adsorption due to the relatively weak interactions involved. Further, many biological materials of interest carry a negative charge at neutral pH and so do not adhere well to solid surfaces that are also negatively charged, such as carbon, which is a commonly used electrode due to its low cost and wide potential window.

Accordingly, there is a need to develop improved techniques of immobilising biomolecules on an electrode surface area, in particular, for use in the development of biosensors that allow for high sensitivity, specificity and rapid detection of target analytes.

This invention aims to obviate or mitigate the existing disadvantages associated with immobilising biomolecules on solid surfaces known in the art.

### SUMMARY OF INVENTION

In a first aspect of the invention, there is provided an electrode comprising:
a conductive element; and
a coating layer formed on at least one surface of the conductive element, wherein the coating layer comprises a polymer having:
   (i) a conjugated system comprising at least two nitrogen atoms; and
   (ii) a hydrophobic region.

The term “conjugated system”, as used herein, refers to a region of the polymer including overlapping orbitals (typically p-orbitals) with delocalised IT electrons. Typically, the conjugated system includes alternating single and multiple bonds. The overlapping p-orbitals bridge the single bonds between adjacent overlapping p-orbitals. Lone pairs, radicals, and carbenium ions may be part of the system. The system may be cyclic, acyclic, or a combination thereof.

Advantageously, delocalisation of cationic charges across the conjugated system provides the coating layer with stabilised positive charges separated by hydrophobic regions. The inventors have surprisingly found that this configuration provides a positively charged surface that can reliably facilitate robust adherence of a negatively charged protein or other biomolecule to the electrode surface.

The conjugated system according to the present invention comprises at least two nitrogen atoms. For example, the conjugated system may comprise a biguanide group represented by Formula (I).

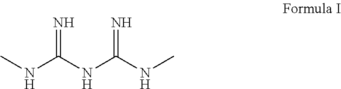

In one example, the coating layer may comprise a polymeric or oligomeric biguanide. Polymeric or oligomeric biguanides are molecules that include multiple biguanide moieties. As understood by those skilled in the art, polymerisation/oligomerisation can generate a population of polymers/oligomers that vary in size, depending on the number of subunits incorporated. The present invention uses polymeric biguanides that average at least three biguanide moieties per molecule. Generally, an average of at least five biguanide moieties per molecule are included. In certain embodiments, the average number of biguanide moieties ranges from 5 to 80, from 5 to 40, from 6 to 27, from 9 to 27, or from 11 to 18. The weight average molecular weight of the polymeric biguanide may exceed 1000 Daltons. For example, in certain embodiments, the weight average molecular weight is from 1000-15,000 Daltons; 1000-8000 Daltons; 1000-5000 Daltons; 1230-5000 Daltons; 1700-5000 Daltons; 2100-5000 Daltons; 2100-3300 Daltons; or 2340-3300 Daltons.

The biguanide moieties are generally joined by a linker that forms the hydrophobic region. In certain examples, a class of polymeric biguanides have the following formula:

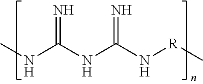

wherein R=an alkyl, a substituted alkyl, an aryl, or a substituted aryl.

For example, the coating layer may comprise polyhexamethylene biguanide (PHMB) or polyaminopropyl biguanide (PAPB). The value of n may be from 4 to 40, or from 4 to 15. In one example, n is 12. Preferably, the average molecular weight of the polymer mixture is from 1100 to 3300 Daltons.

In alternative examples, the coating layer may comprise a bisguanide, such as chlorhexidine or alexidine.

Alternatively, the conjugated system may comprise an imidazolium group. For example, the coating layer may comprise a polymer or oligomer having a repeating unit comprising an imidazolium group. In one example, the coating layer may comprise a polyimidazolium compound as described in WO2021/242174, the contents of which is hereby incorporated by reference in its entirety

The term “conductive element” is used herein to mean an electrically conductive element. Optionally, the conductive element may comprise carbon, for example, graphite, glassy carbon, pyrolitic carbon or screen printed carbon. Advantageously, carbon electrodes are available at low cost and give excellent performance due to a wide range potential window, low resistance, and low residual current. Alternatively, the conductive element may comprise a metal, such as, gold, silver or platinum.

Advantageously, the coating layer adheres to the surface of the conductive element and carries a positive charge in neutral aqueous solutions (i.e. at approximately pH 7.0), which are typically employed for immobilisation of biomolecules on electrode surfaces. On the other hand, many biomolecules of interest carry a negative charge in a neutral aqueous solution. Thus, a biomolecule with a negative charge can be easily and efficiently adsorbed and immobilised on the positively charged coating layer of the conductive element via electrostatic interaction. In other words, the coating layer provides a surface on the conductive element having a positive charge for subsequent attachment of a biomolecule using passive absorption.

Optionally, at least one biological molecule is immobilised on the pre-coated surface of the conductive element. The term “biological molecule” is used in the present application to mean any organic molecule with a biological activity and/or specificity, for example, being capable of specifically binding to a target compound. More specifically, the biological molecule may comprise a nucleic acid molecule, which may be single-stranded or double-stranded (e.g. DNA, RNA or any such chemical 20 substitutes such as PNA etc), amino acid, antibody, peptide, protein, or enzyme. The biological molecule may comprise a whole cell, or part of a cell, a virus, phage, or a micro-organism, or an organelle, or a virus particle etc. For convenience, the terms “biological molecule”, “biomolecule”, “biological element”, and “biological component”, are used herein interchangeably.

Optionally, the biomolecule is streptavidin, avidin or neutravidin. Streptavidin, along with its structural analogues avidin and neutravidin, bonds rapidly with biotin forming one of the strongest known non-covalent interactions. In this way, other biological molecules, such as proteins, enzyme and nucleic acids, can be biotinylated using established protocols known in the art and immobilised onto the streptavidin to provide access to highly sensitive and customisable biosensors.

Accordingly, the electrode may be a working electrode, for example, of an electrochemical biosensor.

In a second aspect of the invention, there is provided a method of manufacturing an electrode, the method comprising:
providing a conductive element; and
forming a coating layer on at least one surface of the conductive element, wherein the coating layer comprises a polymer having a conjugated system comprising at least two nitrogen atoms and a hydrophobic region.

Optionally, the coating layer is formed by contacting the at least one surface of the conductive element with a solution of the polymer. That is, the coating layer may be formed by adsorption of the polymer on the at least one surface of the conductive element. Accordingly, the coating layer may be formed on the surface of the conductive element in a simple and cost-effective manner without the need for elaborate techniques. For example, the coating layer may be formed by incubating the conductive element in a suitable buffer solution containing a suitable concentration of the polymer. Optionally, the solution comprises greater than or equal to about 0.01 w/v of the polymer. The buffer may comprise potassium phosphate solution (Kphos).

After contacting the conductive element with the polymer coating solution, the conductive element may optionally be washed prior to further processing or use. Advantageously, a washing step may remove any excess polymer solution from the electrode surface which may otherwise interfere with subsequent attachment of a biomolecule.

Optionally, the coating layer is dried prior to further processing or use.

Optionally, the method of the second aspect further comprises the precursor steps of:
providing a non-conductive support; and
forming the conductive element on the non-conductive support.

Optionally, the conductive element may be screen printed on the non-conductive support using techniques well known to those skilled in the art. Advantageously, screen printing offers high-mass production of small sized, low cost, disposable, mechanically robust and reliable electrodes in single sensor or array format.

Optionally, the method further comprises the step of attaching a biomolecule to the pre-coated surface of the conductive element. For example, the method may include the step of contacting the coated conductive element with a solution containing the biomolecule. For example, contacting the pre-coated surface of the conducting element with the biomolecule solution may comprise incubating the coated surface in a suitable buffer solution containing a suitable concentration of the biomolecule. Optionally, the buffer comprises Kphos solution. Accordingly, the present invention enables the use of passive absorption to achieve the robust and reliable attachment of a biomolecule to an electrode surface.

In a third aspect of the invention, there is provided an electrochemical sensor comprising at least one electrode according to the first aspect of the invention.

Optionally, the at least one electrode is a working electrode, and the electrochemical sensor further comprises a counter electrode and a reference electrode. The term “working electrode” as used herein refers to the electrode on which the reaction of interest is occurring in the device according to the invention. As is known in the art, the working electrode may be used in conjunction with the counter electrode, and the reference electrode to form a three electrode system. Optionally, the electrochemical sensor comprises a plurality of working electrodes, and a counter electrode and a reference electrode, wherein the plurality of working electrodes each comprise an electrode according to the first aspect of the invention.

In a fourth aspect of the invention, there is provided a use of an electrochemical sensor according to third aspect for detecting an analyte.

The term “analyte” herein refers to a substance that is of interest in an analytical procedure and can comprise chemical or biological analyte molecules. For example, the analyte may be a target biomolecule such as a nucleic acid or protein, or a small molecule. The term “detection” herein not only refers to the qualitative detection of an analyte in a fluid, but also to the quantitative detection of an analyte in a fluid.

Various methods suitable for the electrochemical detection in a biosensor are known in the art. For example, the analyte may be detected using amperometry measurement, impedance measurement or voltammetry measurements.

### BRIEF DESCRIPTION OF FIGURES

The accompanying drawings illustrate presently exemplary embodiments of the disclosure, and together with the general description given above and the detailed description of the embodiments given below, serve to explain, by way of example, the principles of the disclosure.

FIG. 1 shows a schematic sectional view of an electrode;

FIG. 2 shows an exemplary electrochemical sensor;

FIG. 3 shows a graph illustrating the absorption of streptavidin to an electrode having a PHMB coating layer on the surface compared to a carbon electrode without the PHMB coating layer;

FIG. 4 shows a graph illustrating the effect of washing on streptavidin adsorption;

FIG. 5 shows a graph illustrating the effect of using different concentrations of streptavidin on adsorption of streptavidin to the electrode;

FIG. 6 shows a graph illustrating the effect of buffer type and PHMB concentration on adsorption of streptavidin;

FIG. 7 shows a graph illustrating the effect of PHMB concentration in potassium phosphate on adsorption of streptavidin; and

FIG. 8 shows a graph illustrating the absorption of streptavidin to an electrode having a PHMB coating layer on the surface compared to an electrode without the PHMB coating layer and an electrode having a PEI coating layer.

### DETAILED DESCRIPTION

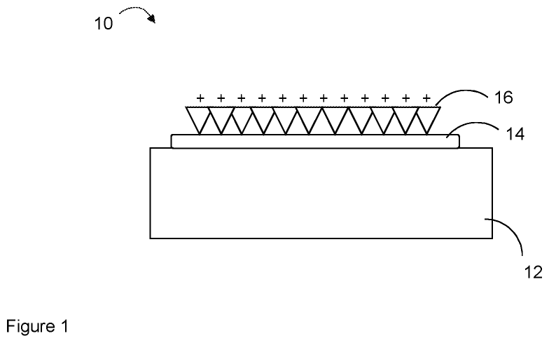

An example structure of an electrode 10 according to the present invention is shown in FIG. 1. The electrode 10 includes a non-conducting support 12, conductive element 14 and a coating layer 16 formed on at least one surface of the conductive element 14. The conductive element 14 may be formed of carbon or other forms of carbon such as graphene, graphite, fullerene, carbon nanotubes, and single/multi-wall carbon nanotubes. Carbon electrodes are commercially available at low cost and give excellent performance due to a wide range potential window, low resistance, and low residual current. However, carbon surfaces carry a negative charge. This can hinder biomolecule adsorption since many biomolecules have isoelectric points that are below pH 7 meaning they are also negatively charged around neutral pH. As a result, charge repulsion occurs between the carbon surface and the biomolecule.

In the present invention, the coating layer, which comprises a polymer having a conjugated system comprising at least two nitrogen atoms and a hydrophobic region, provides a positively charged surface to promote adherence of a negatively charged protein or other biomolecule to the electrode surface.

In some embodiments of the invention, the conjugated system comprises a biguanide group. For example, the coating layer may comprise a polymeric biguanide compound, such as polyhexamethylene biguanide (PHMB). Polymeric biguanides are commercially available or they may be readily prepared by those skilled in the art. The material may be the free base or it may be in the form of a salt. Suitable salts include, but are not limited to, the hydrochloride salt. PHMB is typically known for its use as a disinfectant and antiseptic due to its broad-spectrum antibacterial properties. However, the inventors have surprisingly found that PHMB reliably and robustly adheres to an electrode surface. In this way, a biomolecule with a negative charge can be adsorbed and immobilised on the positively charged PHMB by using electrostatic interaction. Accordingly, the electrode may further comprise a biomolecule layer attached to the coating layer. The biological molecule to be immobilised on the electrode is not particularly limited and can be selected according to the intended use. It will therefore be appreciated that electrodes in accordance the present invention may be used in a wide variety of application fields where it is desirable to functionalise a solid surface with biomolecules.

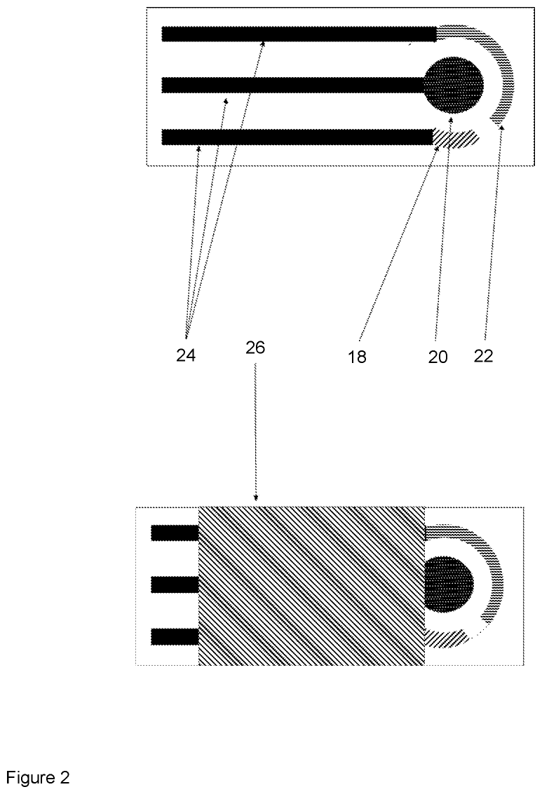

In one example, the electrode of the invention may be used in the fabrication of a biosensor. An example of a biosensor is shown in FIG. 2. The depicted example comprises a reference electrode 18, working electrode 20 and a counter electrode 22 screen printed on a non-conducting solid surface. The sensor further comprises a conductive ink 24 and a dielectric layer 26. Such screen printed electrodes may be obtained using techniques well known to those skilled in the art.

The non-conducting material may be made of any suitable material known in the art, such as alumina, glass, ceramic, or plastic. Reference electrodes are known to the person skilled in the art and may include a standard hydrogen electrode (SHE), a normal hydrogen electrode (HE), a reversible hydrogen electrode (RHE), a saturated calomel electrode (SCE), a copper/copper (II) sulfate electrode (CSE), a silver/silver chloride electrode (Ag/AgCl), a palladium-hydrogen electrode and a dynamic hydrogen electrode. The conductive ink may be formed of carbon or silver and the counter electrode and dielectric layer may be formed of any material that is typical in the art.

The working electrode 20 comprises screen printed carbon, a surface of which is coated with the coating layer. The coating layer may be formed by incubating the conductive element in a suitable buffer solution containing a suitable concentration of the polymer. By way of example, the buffer may comprise potassium phosphate solution (Kphos). A suitable concentration of polymer, e.g. PHMB, may be greater than or equal to about 0.01% (v/v). The electrode may be incubated in the polymer solution for sufficient time and at a suitable temperature to allow the polymer to become attached to the electrode surface. By way of example, a suitable incubation time may be about 45 minutes. The temperature of the incubation may be at room temperature. An average room temperature may generally considered to be in the range of about 20° C. to about 25° C.

After application of the coating layer, the electrode may be washed prior to further processing or use to remove any excess polymer solution from the electrode surface. Excess polymer (i.e. not bound to the electrode surface) may, if present, bind to a biomolecule during subsequent attachment of said biomolecule, thereby preventing a portion of the biomolecule from attaching to the electrode surface. The washing step may be performed with buffer solution and/or water. The electrode may then be dried using any method generally accepted in the art, for example, using passive drying or active drying (e.g., forced air, such as a fan). Drying may be performed at room temperature or at an elevated temperature. In one example, the electrode may be dried in an incubator at 37° C. Dryness of the electrode can be evaluated by any method generally known in the art, for example, to a point of visual dryness.

A suitable biomolecule may then be immobilised on the electrode by contacting the pre-coated surface of the working electrode with a solution containing said biomolecule. The biological molecule may comprise a nucleic acid molecule, which may be single-stranded or double-stranded (e.g. DNA, RNA or any such chemical substitutes such as PNA etc), amino acid, antibody, peptide, protein, or enzyme. The biological molecule may comprise a whole cell, or part of a cell, a virus, phage, or a micro-organism, or an organelle, or a virus particle etc. The biomolecule solution may comprise a suitable buffer solution containing a suitable concentration of the biomolecule. A suitable concentration of the biomolecule in the buffer will depend on the type of biomolecule, and will be known to those skilled in the art. However, by way of example, a suitable concentration of streptavidin, which was used by the inventors to investigate the effectiveness of immobilisation, may be about 1 μg/ml. The electrode may be incubated in the buffer containing the biomolecule for sufficient time and at a suitable temperature to allow the biomolecule to become attached to the coating later. By way of example, a suitable incubation time may be about 45 minutes. The temperature of the incubation may be at room temperature. Following incubation, the electrode surface may then be washed, for example, in fresh buffer solution (not containing any biological molecule) or water. The electrode may then be dried, for example, in an incubator at 37° C.

Biosensors in accordance with the present invention can be used in the detection of an analyte.

Various methods suitable for the electrochemical detection in a biosensor are known in the art. For example, the analyte may be detected using amperometry measurement, impedance measurement or voltammetry measurements. Voltammetric and amperometric techniques are characterised by applying a potential to the working electrode and measuring the current versus the reference electrode. The term voltammetry is used for those techniques in which the potential is scanned over a set potential range. In amperometry, changes in current are monitored in time while a constant potential is maintained at the working electrode with respect to a reference electrode.

The analyte may be a target biomolecule such as a nucleic acid or protein. However, the biomolecule attached to the modified electrode surface can be varied depending on the analyte that is to be detected by the device. Accordingly, biosensors in accordance with the present invention can provide an important tool for sensing analytes in, for example, medicine, in the food industry and for environmental monitoring.

### EXAMPLES

The present invention will be described in greater detail with reference to the following examples. The following examples are for illustrative purposes and are not intended to limit the scope of the invention.

### Materials and Methods

Biotinylated Horseradish Peroxidase was purchased from Thermofisher and Hydroquinone and streptavidin were purchased from Sigma-Aldrich. Polyhexamethylene Biguanide (PHMB; n=12, Polydispersity Index=1.8) was purchased from Arch Biocides Ltd. Polyethylenimine (PEI) was purchased from Merck (#765090).

### Adsorption of PHMB and Streptavidin on to the Electrode Surface

The following Examples were obtained using screen printed carbon sensors obtained via high throughput printing methodology known in the art. Such electrodes are well known in the art and are commercially available, for example, from Metrohm DropSens (#DRP-110).

### Example 1—Preparation of the PHMB-Modified Electrode

The surface of the electrodes were first washed 50 mM Potassium Phosphate buffer, pH 7.4 (Kphos) to remove any surface contaminants. The working electrode was covered with at 0.2% (v/v) solution of PHMB in Kphos and incubated for 45 minutes at room temperature. The working electrode was then washed three times with Kphos to remove excess PHMB solution and dried at 37° C. in an incubator for 45 minutes.

### Example 2—Adsorption Capacity of PHMB-modified Electrode

In the following examples, streptavidin was used to specifically capture biotinylated Horseradish peroxidase (HRP) to exemplify the effectiveness of immobilisation. The electrical signal was measure using amperometric current at −0.35 V against Ag/AgCl upon addition of H₂O₂ to activate HRP and hydroquinone (HQ) as mediator. Other suitable methods for the electrochemical detection of HRP activity are known in the art. Alternatively, an antibody may be immobilised on the electrode surface and detected with an anti-antibody conjugated to HRP using an analogous procedure.

PHMB-modified electrodes were prepared as in Example 1. Streptavidin was adsorbed onto the PHMB-modified surface by contacting the working electrode with a solution of streptavidin in potassium phosphate buffer (50 mM, pH 7.4) at a concentration of 1 μg/ml and incubated at room temperature for 45 minutes.

Excess streptavidin solution was removed, and the electrode washed twice with water. The electrodes were then dried in an incubator at 37° C. for 30 minutes. Using the same procedure, streptavidin was adsorbed onto the working electrode of an otherwise identical sensor that had not undergone the modification step with PHMB. A sensor having a working electrode coated with PHMB that had not been exposed to streptavidin was analysed as a negative control to test for non-specific binding of btHRP to the PHMB layer. The electrocatalytic activity of captured HRP on each sensor was analysed in triplicate using amperometry as described below.

The working electrode was contacted with 50 μl of a detection solution consisting of 1 μg/ml of biotinylated Horseradish Peroxidase in 50 mM Potassium Phosphate Buffer pH 7.4. After 4 minutes, the surface of the working electrode was washed three times with 50 mM Potassium Phosphate Buffer pH 7.4. The sensor was then inserted into a E-Sens reading device (available from Eluceda Limited) for analysis.

50 μl of reading solution consisting of 100 mM Potassium Phosphate, 150 mM Potassium Chloride, 1 mM H₂O₂, 1 mM Hydroquinone was applied to the electrode surface and incubated for 60 seconds. The current return from a chronoamperometric program was measured for 30 seconds at an applied voltage of −0.35V.

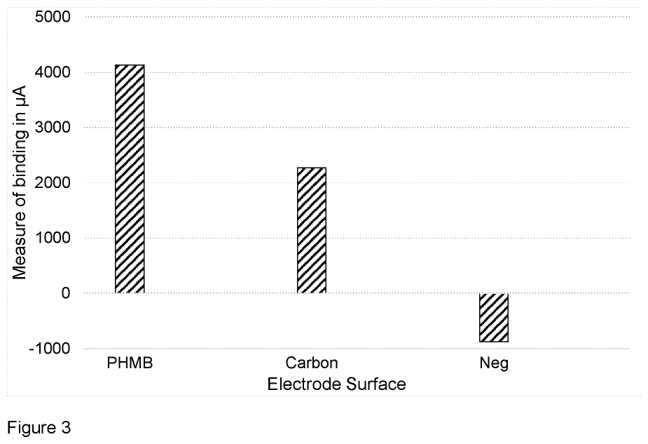

As shown in FIG. 3, an enhanced signal was observed with PHMB-modified electrode, meaning that more streptavidin was bound to the PHMB-modified electrode compared to the unmodified carbon electrode. The absorption of streptavidin to the PHMB-modified electrode was also more reliable compared to absorption to the unmodified carbon electrode, which showed significantly more variability.

### Example 3—Effect of Washing on PHMB Adherence

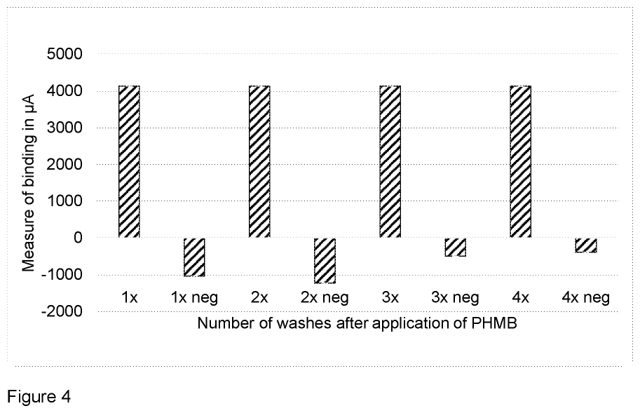

PHMB-modified electrodes were prepared as in Example 1 but with a different number of washing steps after adsorption of PHMB. In each instance, streptavidin was adsorbed onto the surface of the working electrode and detected using the procedure described above for Example 2. The results are shown in FIG. 4 and show that substantially no variation is seen in PHMB adsorption even after excessive washes, indicating that the PHMB is well adhered onto the surface.

### Example 4—Effect of Streptavidin Concentration

Streptavidin was adsorbed onto the surface of PHMB modified electrodes as described above for Example 2 but using different concentrations of streptavidin solution, as detailed in Table 1.

TABLE 1

| | Concentration of streptavidin Solution | |
|---|---|---|
| Example (A) | 2 | ug/ml |
| Example (B) | 1 | ug/ml |
| Example (C) | 0.5 | ug/ml |
| Example (D) | 0.25 | ug/ml |

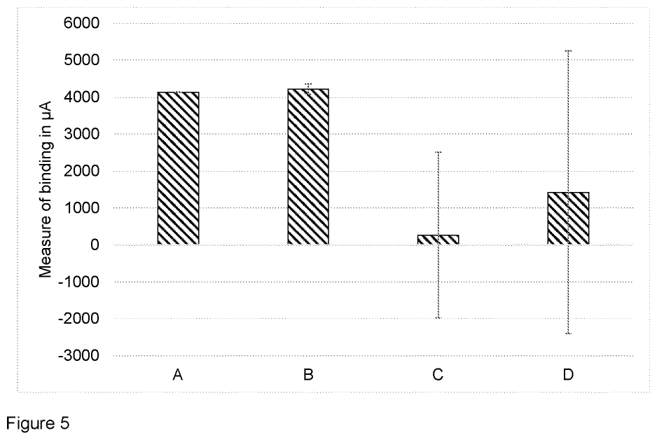

The relative amount of streptavidin adsorbed to the electrodes was determined as described above for Example 2. The results are shown in FIG. 5.

### Example 5—Effect of PHMB Concentration and Buffer Type

PHMB-modified electrodes were prepared as in Example 1 but using different concentration of PHMB and different buffering solution, as detailed in Table 2.

TABLE 2

| | Concentration of PHMB (% v/v) | Buffer |
|---|---|---|
| Example (A) | 2 | Kphos |
| Example (B) | 1.6 | Kphos |
| Example (C) | 0.8 | Kphos |
| Example (D) | 0.2 | Kphos |
| Example (E) | 0.1 | Kphos |
| Example (F) | 0.02 | Kphos |
| Example (G) | 0.01 | Kphos |
| Example (H) | 0.002 | Kphos |
| Example (I) | 0.2 | Tris |
| Example (J) | 0.02 | Tris |
| Example (K) | 0.2 | Hepes |
| Example (L) | 0.02 | Hepes |
| Example (M) | 0.2 | Distilled water |
| Example (N) | 0.02 | Distilled water |
| Control (cA) | 0 | Kphos |
| Control (cB) | 0 | Hepes |
| Control (cC) | 0 | Tris |
| Control (cD) | 0 | Distilled water |

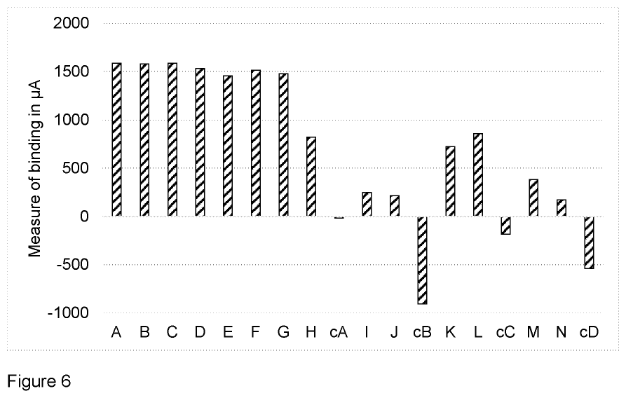

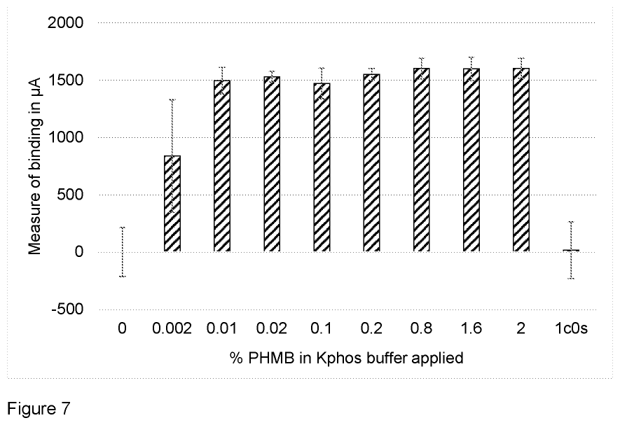

The ability of each of the PHMB coatings to adsorb streptavidin was determined as described above for Example 2. The results are shown in FIG. 6 and demonstrate that a concentration of greater than or equal to 0.01% PHMB in Kphos exhibits the optimum absorption capacity. This can be seen more clearly in FIG. 7, which shows only the results obtained when a solution of PHMB in potassium phosphate buffer was used.

### Example 6—Adsorption Capacity of PHMB-Modified Electrode Vs PEI-Modified Electrode

The adsorption capacity of a PHMB-modified electrode was compared to an equivalent electrode modified with polyethylenimine (PEI). PEI is a polymer with repeating units composed of the amine group and two carbon aliphatic CH₂CH₂ spacers. The amine groups have low pKa values and so carry a positive charge under neutral aqueous conditions.

PHMB-modified electrodes were prepared as in Example 1. PEI-modified electrodes were prepare using the same procedure but using a 0.5 wt. % and a 0.1 wt. % solution of PEI in potassium phosphate buffer (50 mM, pH 7.4).

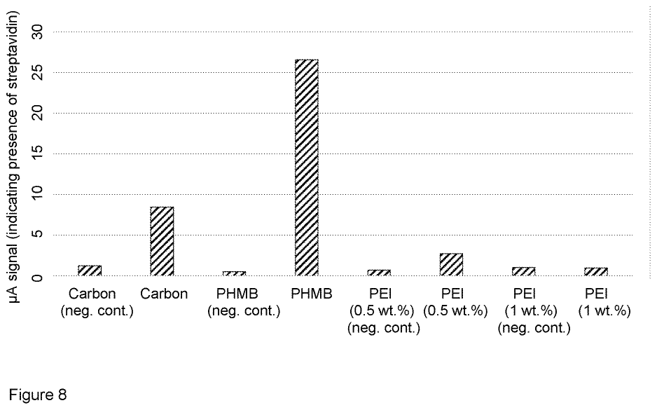

The ability of each of the PHMB coatings to adsorb streptavidin was determined as described above for Example 2. Using the same procedure, streptavidin was adsorbed onto the working electrode of an otherwise identical sensor that had not undergone the modification step with PHMB or PEI. Control sensors having a modified working electrode that had not been exposed to streptavidin were also analysed to test for non-specific binding of btHRP to the PHMB and PEI layers. FIG. 8 shows the adsorption capacity of the various electrode coatings and demonstrates that a PHMB-modified electrode exhibits a stronger absorption capacity for streptavidin than a PEI-modified electrode or a naked, unmodified, electrode. It will therefore be appreciated that the inventors have found a surprisingly effective technique for attaching biomolecules to an electrode surface.

It will be appreciated by persons skilled in the art that the above embodiment has been described by way of example only and not in any limitative sense, and that various alterations and modifications are possible without departing from the scope of the invention as defined by the appended claims.

1. An electrode comprising:
a conductive element; and
a coating layer formed on at least one surface of the conductive element, wherein the coating layer comprises a polymer having:
i. a conjugated system comprising at least two nitrogen atoms, and
ii. a hydrophobic region.

2. The electrode according to claim 1, wherein the conjugated system comprises a biguanide group.

3. The electrode according to claim 1, wherein the conjugated system comprises an imidazolium group.

4. The electrode according to claim 1, wherein the coating layer comprises polyhexamethylene biguanide (PHMB).

5. The electrode according to claim 1, wherein the conductive element comprises carbon.

6. The electrode according to claim 1, wherein the electrode is a working electrode.

7. The electrode according to claim 1, wherein the binding capacity of the coating layer formed on the surface of the conductive substrate is at least 1.5 times the binding capacity of the surface of the conductive substrate.

8. The electrode according to claim 1, wherein at least one protein, antibody, enzyme, nucleic acid or molecule is immobilised on the electrode.

9. The electrode according to claim 8, wherein streptavidin is immobilised on the electrode.

10. A method of manufacturing an electrode for an electrochemical sensor, the method comprising:
providing a conductive element; and
forming a coating layer on at least one surface of the conductive element, wherein the coating layer comprises a polymer having:
i. a conjugated system comprising at least two nitrogen atoms, and
ii. a hydrophobic region.

11. The method according to claim 10, further comprising the steps of:
providing a non-conductive support; and
forming the conductive element on the non-conductive support.

12. The method according to claim 10, wherein the conjugated system comprises a biguanide group.

13. The method according to claim 10, wherein the conjugated system comprises an imidazolium group.

14. The method according to claim 10, wherein the coating layer comprises polyhexamethylene biguanide (PHMB).

15. The method according to claim 10, wherein the conductive element comprises carbon.

16. The method according to claim 10, wherein the coating layer is formed by adsorption of the polymer on the at least one surface of the conductive element.

17. The method according to claim 16, wherein adsorbing the polymer on the at least one surface of the conductive element comprises the steps of:
a) contacting the surface of the conductive element with an aqueous solution of the polymer;
b) washing the surface of the conductive element with water; and optionally
c) drying the surface of the conductive substrate.

18. The method according to claim 17, wherein the aqueous solution of polymer comprises a buffer.

19. The method according to claim 18, wherein the buffer is potassium phosphate.

20. The method according to claim 17, wherein the aqueous solution contains equal to or greater than about 0.01 w/v of the polymer.

21. An electrochemical sensor comprising at least one electrode according to of claim 1.

22. The electrochemical sensor of claim 21, wherein the at least one electrode is a working electrode, and wherein the electrochemical sensor further comprises a counter electrode and a reference electrode.

23. Use of an electrochemical sensor according to claim 21 for detecting an analyte.

24. The use according to claim 23, wherein the analyte is a biomolecule.

25. The use according to claim 23, wherein the analyte is detected by amperometry measurements.
